(19) United States
(12) Patent Application Publication
(10) Pub. No.: US 2021/0137723 A1
(43) Pub. Date: May 13, 2021

(54) A Nasal Device

(71) Applicant: Converse Healthcare Limited, Middlesex (GB)

(72) Inventor: Sanjiv Kanwar, Middlesex (GB)

(73) Assignee: Converse Healthcare Limited, Middlesex (GB)

(21) Appl. No.: 16/768,586

(22) PCT Filed: Dec. 5, 2018
(86) PCT No.: PCT/GB2018/053529

§ 371 Date: May 29, 2020

(30) Foreign Application Priority Data
Dec. 6, 2017 (GB) 1720370.4

(51) Int. Cl. A61F 5/08

(52) U.S. Cl. CPC A61F 5/08; A61M 16/0666

(57) ABSTRACT

A nasal breathing control apparatus has one or more axially extending cylinders having a first end, a second end, a mid-portion and an inner chamber having a first inner end and a second inner end and extending along a longitudinal axis together with a first opening in said first end connected to said inner chamber, a second opening in said second end, connected to said inner chamber and a float having a first end and a second end within the inner chamber and being movable between a first position in which it obturates said first opening and a second position in which it partially obturates the second opening; wherein each of the cylinders is tapered and wherein the cross-section of each of the cylinders comprises a width that decreases from said first end towards said second end of the cylinder.

### INTRODUCTION

The present invention relates to a nasal device and relates particularly but not exclusively to a nasal device for use as a speech aid or teaching device. Such devices may be used to assist a patient in improving the quality and volume that they can speak by controlling the breathing pattern or method of the patient. Such devices are, generally, removably inserted into one or both of the patient's nostrils.

### PRIOR ART

It is well known to provide a patient with a nasal implant for improving the airflow through the nasal passages of a patient. An example of such a device is disclosed in US 2017/0100275 which provides a nasal breathing assist device having one or more open ended tubular elements with a coupling element connecting the ends thereof. The implant is inserted into the nostrils of the patient and helps reduce snoring by increasing airflow through the nasal passageway. The device allows the patient to breath in and out without significant restriction of airflow. A filter may be provided so as to filter air being breathed in.

It is also known to provide filters within implants inserted into the nasal passageways of a patient such as to filter the air that is breathed in by the patient. An example of such a device is disclosed in US 2010/0331777 which provides an implant for insertion into the nasal passageway which includes a screw thread on the outer surface thereof which allows the implant to be screwed into the nasal passageway in a manner which allows the screw thread to engage with the passageway and retain the implant in position. A filter is provided within the implant so as to filter any air being breathed in by the patient.

Whilst the above devices provide perfectly suitable solutions to keeping the nasal passageways open and may also be adapted so as to filter the air being breathed in, they do not lend themselves to being used to control the breathing of a patient and are unable to restrict or eliminate exhaling of air through the nose, as would be required in order to teach a patient to breath in through the nose and breath out through the mouth or to otherwise improve the breathing pattern or method of the patient.

### OBJECT OF THE PRESENT INVENTION

It is an object of the present invention to address the above-mentioned problem by providing a nasal implant which is able to reduce and, if desired, eliminate the possibility of a patient breathing out through the nose whilst allowing the patient to breathe in through the nose without significant restriction.

### STATEMENT OF INVENTION

Accordingly, the present invention provides a nasal breathing control apparatus comprising: one or more axially extending cylinders having a first end, a second end, a mid-portion and an inner chamber having a first inner end and a second inner end and extending along a longitudinal axis; a first opening in said first end connected to said inner chamber; a second opening in said second end, connected to said inner chamber; a float having a first end and a second end within the inner chamber and being movable between a first position in which it obturates said first opening and a second position in which it partially obturates the second opening; wherein each of the one or more axially extending cylinders (12) is tapered and wherein the cross-section of each of the one or more cylinders (12) comprises a width (W) that decreases from said first end (14) towards said second end (16) of the cylinder.

Preferably, said first opening is on said longitudinal axis (X) and said second opening includes one or more openings radially displaced from said central axis (X). Such an arrangement allows airflow around the outside of the float even when the float is blocking a portion of the opening.

In one arrangement said second opening also includes an opening on said central axis (X). Such an arrangement allows for increased airflow through the device when the central opening is not obturated.

The float may include a mid-portion between said first end and said second end and wherein at least said second end comprises a tapered cross-sectional profile having a width (W) which increases from a minimum (W min) at said second end to a maximum (W max) towards said mid portion. Such an arrangement would allow the second end to contact the second inner end of the inner chamber without obturating the one or more radially displaced openings.

Said float may include a mid-portion between said first end and said second end wherein said first end comprises a tapered cross-sectional profile having a width (W) which increases from a minimum (W min) at said first end to a maximum (W max) towards said mid portion. Such an arrangement would allow the first end to contact the first inner end of the inner chamber (20) and obturate the centrally located opening.

The first end of said float comprises a semi-spherical surface. The second end of said float comprises a semi-spherical surface. Semi-spherical surfaces would allow the float to rest in any one of a number of angular positions whilst still performing the desired sealing function.

In the preferred arrangement the float is spherical. Such a shape would allow for the float to settle naturally regardless of angular position.

In one arrangement said inner chamber has an internal diameter (DI) and said float has an external diameter (DE) and wherein said internal diameter (DI) is greater than the external diameter (DE) of the float by an amount G, thereby to provide a flow-path between the float and the internal diameter (DI) of the chamber. In alternative arrangements there may be provided channels (not shown) in the float or the chamber so as to provide the desired flowpath.

In a preferred arrangement the apparatus includes a circumferentially extending projection at the first end of the chamber having an inner diameter D2 which is less than the external diameter DE of the float. Such an arrangement effectively forms the first opening and also helps retain the float within the chamber. Advantageously, said circumferentially extending projection extends a full 360 degrees around the inner surface of the chamber.

Preferably, the arrangement includes two cylinders in which each cylinder has a longitudinal axis (X) extending in parallel to the other and each cylinder is connected to the other cylinder by a bridging portion at the first end of said respective cylinders. Such an arrangement ensures the cylinders are retained together and may also allow for the bridging portion to be used to extract the apparatus from the nostrils of a patient.

Preferably, said cylinder or cylinders comprises a resiliently deformable material. Said projection may also comprises a resiliently deformable material. Still further, said bridging portion may also comprise a resiliently deformable material. In a preferred embodiment, the nasal device comprises a pair of cylinders 12 and a bridging portion, wherein the bridging portion 50 is a gripping means and is formed of a resiliently deformable material with high grip strength.

The apparatus may comprise a plastic material or silicone rubber. Such materials lend themselves to being moulded and the insertion and removal of the float into or out of the cylinders.

In a preferred arrangement the apparatus includes: two cylinders, each cylinder having a longitudinal axis (X) extending in parallel to each other; a bridging portion at the first end of each respective cylinder connecting said cylinders; a circumferentially extending projection within the first end of each cylinder; wherein said two cylinders said bridging portion and said circumferentially extending recess in each cylinder comprise a deformable silicon rubber.

### DRAWINGS

The present invention will now be more particularly described by way of example only with reference to the accompanying drawings, in which:

FIG. 1 is a view of a patient having a nasal device inserted in his nostrils;

FIG. 2 is an isometric view of a nasal device according to aspects of the present invention;

FIG. 3 is a first cross-sectional view of the device in FIG. 2 in which a portion of the device has been omitted so as to assist the clarity of the drawing;

FIG. 4 is a second cross-sectional view of the device of FIG. 2 and shows a first arrangement of the device when the patient is attempting to breath out;

FIG. 5 is a third cross-sectional view of the device in FIG. 2 and shows a second arrangement of the device when the patient is breathing in; and

FIG. 6 is a still further cross-sectional view of the device in FIG. 2 but illustrating two alternative forms of movable element.

### SPECIFIC DESCRIPTION

Referring now to the drawings in general but particularly to FIGS. 1 and 2, a patient 1 may be provided with a breathing control nasal device 10 such as that shown in FIG. 2 if the patient is desirous of improving their breathing technique or suffers from breathing difficulties which could be corrected using such a device 10. The present device 10 is designed to allow the patient 1 to breathe in through their nostrils but prevents them from breathing out through their nostrils such as to encourage the patient to breathe out through their mouth.

The device comprises one or more one or more axially extending cylinders 12 having a first end 14, a second end 16, a mid-portion 18 and an inner chamber 20. As best seen in FIGS. 3 to 5, the or each chamber 20 has a first inner end 20a and a second inner end 20b and extending along a longitudinal axis X. A first opening 22 in said first end 14 connects to said inner chamber 20 whilst a second opening 24 in said second end 16 also connects to said inner chamber 20, such as to allow air to be drawn through the device 10 as will be explained in detail later herein. A float 26 having a first end 26a and a second end 26b is provided within the inner chamber 20 and is movable between a first position FP in which it obturates (blocks to prevent flow) said first opening 22 but does not obturate the second opening 24 and a second position SP in which it partially obturates the second opening 24 but does not obturate the first opening 22. FIGS. 4 and 5 illustrate these two positions of the float within the chamber.

The float 26 may take any one of a number of forms and the first and second openings 22, 24 may be arranged to suit the shape of the float so long as the function of being able to obturate the first opening 22 when placed thereagainst (as in FIG. 4) and allow for airflow through the second opening 24 when placed thereagainst (as in FIG. 5) is facilitated. The preferred arrangement of the openings 22 and 24 is shown in FIGS. 3 to 5 and from which it will be appreciated that opening 22 best comprises a single opening and is, preferably, positioned on the central line X whilst the second opening 24 comprises a plurality of openings 24a, 24b, 24c, some or all of which are radially displaced from said central axis X, for reasons that will become apparent later herein. Indeed, it is possible to eliminate any second opening 24c that may lie on the central axis X as it is the radially displaced openings 24a, 24b which are primarily involved in the functioning of the apparatus 10.

The general requirement for the float or floats 26 that it or they is able to move within the chamber or chambers 20 between the first and second openings 22, 24 such as to interact with said openings 22, 24 in order to control or prevent the flow of air therethrough. For reasons of brevity, the remaining description will refer to the floats and chambers in the singular but it will be appreciated that the description will apply equally to singular or plural of said items. Said float 26 includes a mid-portion 26m between said first end 26a and said second end 26b and at least said second end 26b comprises a tapered cross-sectional profile having a width W which increases from a minimum W min at said second end 26b to a maximum W max towards said mid portion 26m, thereby to allow the second end 26b to contact the second inner end 20b of the inner chamber 20 without obturating the one or more radially displaced openings 24a, 24b. Said first end 26a comprises a tapered cross-sectional profile having a width W which increases from a minimum W min at said first end (26a) to a maximum W max towards said mid portion 26m, thereby to allow the first end 26b to contact the first inner end 20a of the inner chamber 20 and obturate the centrally located opening 22. In some embodiments of the float the first end is shaped such as to positively extend into said first opening 22 (left hand arrangement of FIG. 6) whilst on other arrangements the float 26 simply sits on the lip 22a of the opening but does not extend significantly therein. In the preferred arrangement of the float 26, and as shown in FIG. 5, the first end 26a of said float 26 comprises a semi-spherical surface 26e whilst the second end 26b of said float 26 may also comprise a semi-spherical surface 26f. The semi-spherical form or forms allow the float 26 to readily self-centralise on the first opening 22 and a central second opening 24c, if provided. The most convenient form of the float 26 is a spherical float (as shown) as such are readily available as commercial products and their spherical shape avoids possible problems of alignment as may be present in tubular or elongate shaped floats.

FIG. 6 illustrates two alternative forms of float 26 which may be used as an alternative to the spherical arrangements of FIGS. 4 and 5. The float 26 on the left-hand-side of FIG. 6 is formed as a tubular float with a mid-portion 26m which extends axially in the general direction of axis X such as to provide an elongate surface for interaction with the inner sidewalls 20s of the chamber 20 in a sliding manner which may assist with the alignment of the float centrally or assist simply with preventing the float 26 sticking in any one position. The ends of the float may comprise semi-spherical surfaces 26e as shown on the left-hand arrangement of FIG. 6 or may comprise proper tapered surfaces 26b with pointed ends, as shown on the right-hand side of FIG. 6. It will be appreciated that the end surface of the tapered arrangement tapers from a maximum width WMAX at the sides of the float to a minimum width WMIN at the tip of the float. The semi-spherical ends help with centralising the float 26 within the chamber once the float contacts one or other of the inner ends 26a, 26b.

It will be appreciated that the float 26 must be allowed to move freely within the chamber 20 and a gap G must be provided between the float 26 and the walls 20w of the chamber 20. Consequently, it is preferred to arrange the chamber 20 and float 26 such that said inner chamber 20 has an internal diameter DI and said float 26 has an external diameter DE and wherein said internal diameter DI is greater than the external diameter DE of the float by an amount G, thereby to provide a flow-path, represented by arrow P, between the float 26 and the walls 20w of the chamber 20, which may be used for the passage of air through the chamber as and when required.

It will be appreciated that the float may be retained within the chamber 20 by any one of a number of conventional means but a particularly suitable means is a circumferentially extending projection 40 at the first end 20a of the chamber 20 having an inner diameter D2 which is less than the external diameter DE of the float 26. Such a projection 40 effectively acts to form the first opening 22 and also retain the float 26 within the chamber. It will be appreciated that the projection may extend around the inner surface 20w of the chamber and may actually extend a full 360 degrees around the inner surface should that be desired. The projection may comprise a separately formed inner ring (not shown) inserted into the chamber 20 and secured thereto or it may comprise a projection 40 formed integrally with the cylinder itself 12.

Preferably said cylinder or cylinders can be shaped such that they are complementary in shape to the nasal passage of a nostril. Each of the one or more axially extending cylinders 12 of the nasal device may be adapted to have an external shape that securely fits the shape and/or dimensions of the human nasal passage. The cylinder(s) 12 can be shaped as cones, conical frustums, tapered cylinders, bullet shapes or funnels to aid insertion of the nasal device into the nasal passage of the nostril and/or to ensure a secure fit for the device in the nostril. Upon insertion of the nasal device into the nasal passage, the first end 14 of the cylinder 12 is located at the nostril entrance, and the second end 16 is located in the inner cavity of the nasal passage, thereby allowing airflow from the external environment into the nostril via the first opening 22 of the cylinder 12 and into the nasal passage way via the second opening 24 of the cylinder 12.

In a preferred embodiment, each of the one or more axially extending cylinders 12 of the nasal device is adapted to have an external shape that securely fits the shape and/or dimensions of the human nasal passage and wherein the first end 14 of the cylinder 12 is adapted to fit the nostril entrance to the nasal passage and the second end 16 of the cylinder 12 is adapted to fit the nasal passage. It will be appreciated that the human nasal passage can vary in width and length and there are therefore numerous shapes of cylinder that will be suitable for insertion into the nasal passage.

In a preferred embodiment, each of the one or more axially extending cylinders 12 of the nasal device is tapered and the cross-section of each of the one or more cylinders 12 comprises a width (W) that decreases from said first end 14 towards said second end 16 of the cylinder. As illustrated in FIG. 3, the cross-sectional profile of the cylinder 12 has a maximum width (W₂) at the first end 14 of the cylinder and the width decreases (i.e. tapers) to a minimum width (W₁) at the second end 16 of the cylinder thereby aiding insertion of the nasal device into the nasal passage. The width of the cross-section at the first end 14 of the axially extending cylinder 12 is therefore greater than the width of cross-section at the second end 16 of the axially extending cylinder. In one embodiment, the second end 16 of each of the one or more axially extending cylinders 12 is dome shaped. In one embodiment, the second end 16 of each of the one or more axially extending cylinders 12 is blunted.

In one embodiment, each of the one or more axially extending cylinders 12 of the nasal device is bullet shaped, wherein the cylinder 12 comprises a first end 14 and a second end 16 and wherein the second end 16 is the tapered dome portion the bullet shaped cylinder. It is well understood that a bullet shape comprises a cylindrical portion and a generally tapered domed end portion.

The more complete form of the present invention comprises a pair of cylinders 12 connected to each other by a bridging portion 50 at the first end of said respective cylinders 12. This bridging portion 50 extends between the nostrils when the apparatus 10 is inserted into the two nostrils of a patient and retains the cylinders 10 together as one device. The bridging portion 50 may also be used as a grip when extracting the cylinders 12 from the patient's nostrils.

One or more of the components of the device 10 may be moulded or otherwise formed from plastics material such as to form separate or joined components. Deformable plastic may be used and it has been found that resiliently deformable silicon is particularly suitable for this application. The cylinders 12, bridging portion 40 and circumferentially extending projection 40 may be formed as one by, for example, moulding. Deformable plastic or resiliently deformable silicone has a number of advantages when used in association with the present invention. Firstly, the cylinders 12 may more easily be compressed to fit within the patient's nostrils and if resiliently deformable plastic or resiliently deformable silicone is used then the material will actually grip the inside of the patient's nostrils, thus reducing the chances of the device 10 being removed inadvertently. In a preferred embodiment, the nasal device comprises a pair of cylinders 12 and a bridging portion, wherein the bridging portion 50 is a gripping means and is formed of a resiliently deformable material with high grip strength. In one embodiment, the material can be silicone or plastic. The bridging portion 50 acting as a gripping means clamps the nasal device to the septum of a human nose such that the two cylinders are held in place in the two nostrils of the nose (one cylinder per nostril), thereby providing a secure fit to the nose for the user of the nasal device.

Still further, resiliently deformable plastics and, in particular, silicone allow the cylinders 12 and axially extending projection 40 to be formed as a single item of moulded construction as the flexibility and resilience of the material once moulded will allow it to be removed from the mould, despite the mould needing an inner form to create the chamber 20, as the deformation of the deformable plastic or silicon may be used to advantage and it may be deformed during extraction from the mould. The circumferentially extending projection 40 if formed from resiliently deformable plastic or silicone would also allow for the insertion of the float 26 into the chamber 20 as the projections 40 might simply deform out of the way during insertion of the float 20. This effect is enhanced further if the cylinders 12 are also formed of the same resiliently flexible plastic or silicone as they too would stretch or deform to accommodate the insertion of the float 26 into the chamber 20.

1-20. (canceled)

21. A nasal breathing control apparatus comprising:
one or more axially extending cylinders having a first end, a second end, a mid-portion and an inner chamber having a first inner end and a second inner end and extending along a longitudinal axis (X);
a first opening in said first end connected to said inner chamber;
a second opening in said second end, connected to said inner chamber;
a float having a first end and a second end within the inner chamber and being movable between a first position FP in which it obturates said first opening and a second position SP in which it partially obturates the second opening; wherein each of the one or more axially extending cylinders is tapered and wherein the cross-section of each of the one or more cylinders comprises a width (W) that decreases from said first end towards said second end of the cylinder.

22. The apparatus as claimed in claim 21, wherein the cross-sectional profile of the cylinder has a maximum width (W₂) at the first end of the cylinder and the width decreases to a minimum width (W₁) at the second end.

23. The apparatus as claimed in claim 21, wherein each of the one or more axially extending cylinders of the nasal device is bullet shaped and wherein the second end of the cylinder is positioned at the tapered portion the bullet shaped cylinder.

24. An apparatus as claimed in claim 21, in which said first opening is on said longitudinal axis (X) and said second opening includes one or more openings radially displaced from said central axis (X).

25. An apparatus as claimed in claim 24 in which said second opening also includes an opening on said central axis (X).

26. An apparatus as claimed in claim 24, wherein said float includes a mid-portion between said first end and said second end and wherein at least said second end comprises a tapered cross-sectional profile having a width (W) which increases from a minimum (W min) at said second end to a maximum (W max) towards said mid portion, thereby to allow the second end to contact the second inner end of the inner chamber without obturating the one or more radially displaced openings.

27. An apparatus as claimed in claim 24, wherein said float includes a mid-portion between said first end and said second end wherein said first end comprises a tapered cross-sectional profile having a width (W) which increases from a minimum (W min) at said first end to a maximum (W max) towards said mid portion, thereby to allow the first end to contact the first inner end of the inner chamber and obturate the centrally located opening.

28. An apparatus as claimed in claim 21, wherein said first end of said float comprises a semi-spherical surface.

29. An apparatus as claimed in claim 21, wherein said second end of said float comprises a semi-spherical surface.

30. An apparatus as claimed in claim 21, wherein said float is spherical.

31. An apparatus as claimed in claim 21, wherein said inner chamber has an internal diameter (D1) and said float has an external diameter (DE) and wherein said internal diameter (D1) is greater than the external diameter (DE) of the float by an amount G, thereby to provide a flow-path between the float and the internal diameter (D1) of the chamber.

32. An apparatus as claimed in claim 21, and including a circumferentially extending projection at the first end of the chamber having an inner diameter (D2) which is less than the external diameter (DE) of the float.

33. An apparatus as clamed in claim 32, and wherein said circumferentially extending projection extends a full 360 degrees around the inner surface of the chamber.

34. An apparatus as claimed in claim 21, and including two cylinders, each cylinder having a longitudinal axis (X) extending in parallel to each other and each cylinder being connected to the other cylinder by a bridging portion at the first end of said respective cylinders.

35. An apparatus as claimed in claim 21, wherein said cylinder comprises a resiliently deformable material.

36. An apparatus as claimed in claim 21, wherein said projection comprises a resiliently deformable material.

37. An apparatus as claimed in claim 21, wherein said bridging portion comprises a resiliently deformable material.

38. An apparatus as claimed in claim 21, wherein said apparatus comprises a plastic material.

39. An apparatus as claimed in claim 21, wherein said apparatus comprises silicon rubber.

40. An apparatus as claimed in claim 21, and comprising:
two cylinders, each cylinder having a longitudinal axis (X) extending in parallel to each other;
a bridging portion at the first end of each respective cylinder connecting said cylinders;
a circumferentially extending projection within the first end of each cylinder; and
wherein said two cylinders said bridging portion and said circumferentially extending recess in each cylinder comprise a deformable silicon rubber.
